(19) United States
(12) Patent Application Publication
(10) Pub. No.: US 2006/0073112 A1
(43) Pub. Date: Apr. 6, 2006

(54) Method of preventing hydrogen sulfide odor generation in an aqueous medium

(76) Inventor: Wilson K. Whitekettle, Jamison, PA (US)

Correspondence Address: DUANE MORRIS, LLP;IP DEPARTMENT, 30 SOUTH 17TH STREET, PHILADELPHIA, PA, 19103-4196, US

(21) Appl. No.: 10/957,366

(22) Filed: Oct. 1, 2004

(51) Int. Cl. A61L 9/01

(52) U.S. Cl. 424/76.1

(57) ABSTRACT

The present invention relates to a fast acting chemical treatment for preventing the generation of hydrogen sulfide odor by the microbial metabolic activities of sulfate reducing bacteria. Specifically, the invention relates to a method for preventing hydrogen sulfide odor generation in a sulfur species-containing aqueous medium, which includes adding to the aqueous medium an effective amount for the purpose of a sulfide generation inhibitor treatment selected from the group consisting of ionophores, 2-mercaptobenzothiazole, alkanol amines, and mixtures thereof.

### FIELD OF THE INVENTION

The present invention relates to a fast-acting, aqueous phase chemical treatment for preventing the generation of hydrogen sulfide odor in an aqueous medium.

### BACKGROUND OF THE INVENTION

The reactivity between various aldehydes and sulfidic compounds (H₂S, mercaptans, etc.) has been known in the art for some time. For example, Marks in U.S. Pat. No. 1,991,765 discloses a method of reacting hydrogen sulfide and an aldehyde in an aqueous solution having a pH between 2 and 12 at a temperature between substantially 20° C. and 100° C. After Marks' disclosure in 1935, many patents appeared disclosing the use of aldehydes during acid cleaning of iron sulfide deposits, including U.S. Pat. Nos. 2,606,873; 3,514,410; 3,585,069; 3,669,613; 4,220,550; 4,289,639; and 4,310,435. Consumption of the hydrogen sulfide liberated by acidification of sulfide-containing deposits increased the safety of such operations. Decreased corrosivity of the aldehyde-containing acids is also disclosed in the prior art, sometimes with the addition of ancillary corrosion inhibitors.

Menaul in U.S. Pat. No. 2,426,318 discloses a method of inhibiting the corrosive action of natural gas and oil containing soluble sulfides on metals by utilizing an aldehyde, preferably formaldehyde.

Roehm in U.S. Pat. No. 3,459,852 discloses a process for deodorizing and reducing the biochemical demand of an aqueous solution which contains at least one compound of hydrogen sulfide and compounds containing the —SH group. Roehm's process comprises mixing the solution with a sulfide-active alpha, beta unsaturated aldehyde or ketone in an amount sufficient to form a sulfur-containing reaction product of the sulfide active aldehyde or ketone. Two such sulfide-active compounds disclosed by Roehm are acrolein and 3-buten-2-one.

Formaldehyde, formaldehyde with SO₃⁻², and acrolein are all commercially used hydrogen sulfide (H₂S) scavengers. However, formaldehyde produces a solid reaction product and reverts readily to formaldehyde and free H2S. Acrolein is more expensive than formaldehyde as well as extremely toxic and dangerous to handle. The use of SO3-2 with formaldehyde eliminates the re-release of H₂S but not solids formation.

Despite the prior art approaches to H₂S scavenging, the provision of a product that can prevent the H₂S formation microbiologically (rather than removal of H₂S after it is formed) is highly desirable, since it is a more effective way of controlling H₂S odor and preventing corrosion and related problems. Existing H₂S prevention methods that include utilizing biocides to kill microbes such as Sulfate Reducing Bacteria (SRBs) are non-selective and will kill other microorganisms, and may be detrimental to the wastewater remediation processes. These concerns are effectively negated by the utilization of the hydrogen sulfide prevention methods of the present invention, often combined with an H₂S scavenging component chemical.

### SUMMARY OF THE INVENTION

The present invention relates to a fast acting chemical treatment for preventing the generation of hydrogen sulfide odor by the microbial metabolic activities of sulfate reducing bacteria. Specifically, the invention relates to a method for preventing hydrogen sulfide odor generation in a sulfur species-containing aqueous medium, which comprises adding to the aqueous medium an effective amount for the purpose of a sulfide generation inhibitor treatment selected from the group consisting of ionophores (e.g., monensin), 2-mercaptobenzothiazole, or alkanol amines (e.g., monoethanolamine).

### DESCRIPTION OF THE PREFERRED EMBODIMENTS

The present invention utilizes chemicals which can act as redox potential buffers to treat a liquid medium containing microorganisms (such as, but not limited to sulfate reducing bacteria, or SRB), which can generate H₂S odor by converting sulfur species of higher oxidation states to sulfides (S²⁻). By the addition of a small but effective amount of such agents to the liquid (e.g., wastewater, industrial cooling water or water/hydrocarbon emulsion) medium, the sulfide is not microbiologically produced. These agents are introduced in two different ways (alone or in combination) in order to inhibit the formation of odor causing volatile sulfides. They function by inhibiting the intracellular biological processes that allow certain microorganisms, (e.g. anaerobic sulfate reducing bacteria) to biochemically reduce oxidized forms of sulfur to sulfides, particularly the gas hydrogen sulfide. These agents specifically interfere with the sulfate reduction process and do not inhibit beneficial microbial processes, nor cause cell death of the general microbial population, or the sulfate reducing bacteria themselves.

Since H₂S is a highly corrosive gas, preventing H₂S from forming in systems such as cooling systems and storage tanks also provides an effective means of corrosion control. Further, in a preferred embodiment, the present invention relates to a method for preventing hydrogen sulfide odor generation in a hydrogen sulfide-containing aqueous medium, which comprises adding to the aqueous medium an effective amount for the purpose of one of the test compounds cited. About 5-50 ppm of the treatment is preferably added to the aqueous medium, with an amount of treatment of about 25 ppm being particularly preferred.

It has been found that the treatment of the present invention is effective and fast-acting in preventing the generation of hydrogen sulfide odor in an aqueous medium with a pH of from about 6.0 to 8.5.

The invention will be further illustrated by the following examples, which are included as being illustrations of the invention and which should not be construed as limiting the scope thereof.

### EXAMPLES

The test protocol was as follows: 100 mL serum bottles were filled with synthetic nutrient medium, then stoppered, capped, and autoclaved, and allowed to cool to at least 35° C. Using a 1 mL syringe with needle, the treatment was added at desired dosages to prepared serum bottles (triplicates of each dosage). No treatment was added for baseline controls. Nine baseline serum bottles were used.

Next, a 10¹⁰ cells/ml concentration of the sulfate reducers was prepared (using autoclaved synthetic nutrient medium) inside an anaerobic chamber. Using a 1 ml syringe with needle, 0.1 ml of the prepared sulfate reducers culture was added to each serum bottle. The bottles were then incubated at the desired temperature for 48 hours.

The baseline dissolved sulfide levels were then measured to determine if the samples were ready for efficacy analysis. Using a 30 mL syringe with needle, 30 mL of sample was removed from each serum bottle. About 25 mL of the sample was added to a 30 mL bottle containing 0.125 mL zinc acetate solution, followed by adding 1 mL of 1N NaOH. The sulfide in the fixed sample was then measured. If the control sample sulfide levels were >10 ppm, then the whole series of test vials was evaluated for generated sulfide. The remaining 5 mL of sample in the syringe was used for a final pH determination.

As shown in Table I below, the treatments of the present invention are non-biocidal in nature, i.e., sulfate reducing bacteria viabilities are not irreversibly affected by the treatments. Furthermore, the treatments of the present invention do not result in a significant alteration of medium pH at appropriate dosages. Note that at application dosages for all chemical treatments listed, approximately 100% inhibition of H₂S production was achieved with 25-100 ppm of the test compounds.

TABLE I
Effectiveness of Inhibitors of Hydrogen Sulfide Generation

| | | Sulfide Formation | | | |
|---|---|---|---|---|---|
| | | Level | | | |
| | Inhibitor Dose | Sample | | | |
| Sample | Conc. (ppm) | A | B | C | SRB/ml |
| Control 1-3 | 0 | 3+ | 3+ | 3+ | 100,000 |
| Control 4-6 | 0 | 3+ | 3+ | 3+ | 100,000 |
| Control 7-9 | 0 | 3+ | 3+ | 3+ | 100,000 |
| 2-mercaptobenzothiazole | 25 | 0 | 0 | 0 | 10,000 |
| 2-mercaptobenzothiazole | 50 | 0 | 0 | 0 | 100,000 |
| | | Sulfide Formation | | | |
| | | Level ¹ | | | |
| | Inhibitor Dose | Sample | | | |
| Sample | Conc. (ppm) | A | B | C | SRB/ml |
| 2-mercaptobenzothiazole | 100 | 0 | 0 | 0 | 10,000 |
| monoethanolamine | 25 | 1+ | 1+ | 1+ | 100,000 |
| monoethanolamine | 50 | 1+ | 1+ | 0 | 100,000 |
| monoethanolamine | 100 | 0 | 0 | 0 | 100,000 |
| monensin | 25 | 1+ | 1+ | 0 | 1,000,000 |
| monensin | 50 | 0 | 0 | 0 | 100,000 |
| monensin | 100 | 0 | 0 | 0 | 10,000 |

¹ 3+ = >20 ppm Sulfide
2+ = 1.1-20 ppm Sulfide
1+ = 0.1-1.0 ppm Sulfide
0 = <0.1 ppm Sulfide

The treatment of the present invention was also found to be stable with time and temperature, ranging from about 3° C. to about 35° C., although the present invention is expected to be effective within a temperature range of from about 0° C. to about 50° C.

While the present invention has been described with respect to particular embodiments thereof, it is apparent that numerous other forms and modifications of the invention will be obvious to those skilled in the art. The appended claims and this invention generally should be construed to cover all such obvious forms and modifications, which are within the true spirit and scope of the present invention.

What is claimed is:

1. A method for preventing hydrogen sulfide odor generation in a sulfur species-containing aqueous medium, which comprises adding to the aqueous medium an effective amount for the purpose of a sulfide inhibitor treatment selected from the group consisting of an ionophore, 2-mercaptobenzothiazole, an alkanolamine and mixtures thereof.

2. The method as recited in claim 1, wherein the ionophore is monensin.

3. The method as recited in claim 1, wherein from about 5-50 ppm of the treatment is added to the aqueous medium.

4. The method as recited in claim 1, wherein the addition is conducted at a temperature of from about 0° C. to about 50° C.

5. The method as recited in claim 3, wherein about 25 ppm of the treatment is added to the aqueous medium.

6. The method as recited in claim 1, wherein said aqueous medium has a pH of about 6.0-8.5.

7. The method as recited in claim 1, wherein said aqueous medium comprises a wastewater or industrial cooling water medium.

8. The method as recited in claim 1, wherein the alkanolamine is monoethanolamine.

9. The method as recited in claim 4, wherein the addition is conducted at a temperature of from about 3° C. to about 35° C.

10. A method for controlling corrosion in a sulfur species-containing aqueous medium, which comprises adding to the aqueous medium an effective amount for the purpose of a sulfide inhibitor treatment selected from the group consisting of an ionophore, 2-mercaptobenzothiazole, an alkanolamine and mixtures thereof.

11. The method as recited in claim 10, wherein the ionophore is monensin.

12. The method as recited in claim 10, wherein from about 5-50 ppm of the treatment is added to the aqueous medium.

13. The method as recited in claim 10, wherein the addition is conducted at a temperature of from about 0° C. to about 50° C.

14. The method as recited in claim 12, wherein about 25 ppm of the treatment is added to the aqueous medium.

15. The method as recited in claim 10, wherein said aqueous medium has a pH of about 6.0-8.5.

16. The method as recited in claim 10, wherein said aqueous medium comprises a wastewater or industrial cooling water medium.

17. The method as recited in claim 10, wherein the alkanolamine is monoethanolamine.

18. The method as recited in claim 13, wherein the addition is conducted at a temperature of from about 3° C. to about 35° C.
